(19) United States
(12) Patent Application Publication
(10) Pub. No.: US 2005/0287523 A1
(43) Pub. Date: Dec. 29, 2005

(54) Functionalized platform for individual molecule or cell characterization

(75) Inventors: Sonia E. Letant, Livermore, CA (US); Anthony W. van Buuren, Livermore, CA (US); Louisa J. Hope-Weeks, Lubbock, TX (US); Louis J. Terminello, Danville, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 11/140,391

(22) Filed: May 26, 2005

Related U.S. Application Data
(60) Provisional application No. 60/576,154, filed on Jun. 1, 2004.

(51) Int. Cl. C12Q 1/70; C12M 1/34

(52) U.S. Cl. 435/5; 435/287.1

(57) ABSTRACT

A system for characterization of a single molecule or cell sample by directing a beam onto the sample to produce energy emanating from the sample. A periodic sample holder with through-pores containing the sample is positioned to receive the beam. At least one pore is provided in the sample holder for holding the sample. The energy emanating from the sample is detected by a detector. The sample holder can be used to study individual molecules, viruses, or cells by various techniques including x-ray diffraction, chemical analysis, optical or electron microscopy, or electrochemistry.

### CROSS-REFERENCE TO RELATED APPLICATIONS

This application claims the benefit of U.S. Provisional Patent Application No. 60/576,154 filed Jun. 1, 2004 by Sonia E. Létant, Anthony W. van Buuren, Louisa J. Hope-Weeks, and Louis J. Terminello; titled “Functionalized Silicon Platform for Individual Bio-Molecule Characterization.” U.S. Provisional Patent Application No. 60/576,154 filed Jun. 1, 2004 and titled “Functionalized Silicon Platform for Individual Bio-Molecule Characterization” is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to characterization and more particularly to a functionalized platform for individual molecule or cell characterization.

2. State of Technology

The “State of Technology” as the present invention relates to protein crystallography includes the following: protein crystallography is a technique that allows the determination of the 3-dimensional structures of biological macro-molecules. Knowledge of the atomic structure of macromolecules such as enzymes, DNA binding proteins and viruses is progressively leading to a better understanding of the chemical reactions which take place in living organisms, how proteins are produced and how genetic information is forwarded. It also provides a basis for drug, vaccine and treatment design.

The main obstacle to the crystallography technique is that only macro-molecules which crystallize can be studied. But although NMR spectroscopy has provided the structures of small proteins from samples in solution, crystallographic methods remain the most successful and used means of determining the atomic structure of large proteins and viruses.

Crystallization constitutes the rate limiting step in protein crystallography. Several methods of crystallization are now well established such as micro-batch crystallization and vapor diffusion but application of these methods is still very much trial and error. Crystallization of a newly isolated protein can take weeks, months or even years if at all.

In order to overcome this problem, a considerable amount of research is now dedicated to the development of algorithms that will allow the inversion of X-ray pulses diffracted by single molecules and a proof of concept has recently been achieved with a pattern of 50 nm colloidal gold beads placed on a silicon nitride membrane. But another step on the road of the achievement of this scientific milestone is to build a platform that will allow single molecules to be presented in the diffractive beam (X-ray, electrons or neutrons) with a controlled position and orientation, synchronized with the X-ray pulses.

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for characterization of a sample by directing a beam onto the sample to produce energy emanating from the sample. The energy emanating from the sample is detected by a detector. A sample holder is positioned to receive the beam. The sample holder contains at least one pore and the pore is functionalized to accommodate one molecule per hole or one cell per hole. In one embodiment at least one through hole is fabricated on a rigid platform for holding the sample. In one embodiment, the diffraction pattern from the sample is detected by a detector. In one embodiment, an apparatus for characterization of a sample comprises a source for directing a diffractive beam onto the sample to produce a diffraction pattern, a sample holder, at least one pore in said sample holder for holding the sample, and a detector for detecting the diffraction pattern.

The present invention has numerous uses. For example the present invention has use for crystallographic structure of proteins and viruses in either the dry or hydrated state. The present invention has use for investigation of the effect of a single or of multiple linkers on protein conformation, investigation of the effect of solution parameters such as pH and salt concentration on protein conformation, in situ binding experiments on systems such as ssb (single strand DNA binding) protein—DNA, and investigation of protein complex formation. The present invention also has use for optical and electronic microscopy, luminescence, electrochemistry, current blockade measurements and Coulter-counting, Secondary Ion Mass Spectrometry (SIMS, ToF SIMS, Nano-SIMS), and Energy Dispersive X-ray Spectroscopy (EDS).

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1A illustrates a system for bio-molecule characterization.

FIG. 1B shows the functionalized platform for individual bio-molecule characterization portion of the system for bio-molecule characterization in greater detail.

FIG. 2 shows a top view of a protein holder.

FIG. 3 shows a side cross section view of a protein holder.

FIG. 4 shows a side cross section view of a protein holder with hydrated phase moved under an X-ray beam.

FIG. 5 shows a FESEM cross section of a pore having a 500 nm pore diameter in a silicon membrane.

FIG. 6 shows a close up on one pore in a silicon membrane.

FIG. 7 shows a top view of various through apertures prepared by FIB drilling in a silicon membrane.

FIG. 8 shows a top view of another set of through apertures prepared by FIB drilling in a silicon membrane.

FIG. 9A shows a pore grid structure.

FIG. 9B shows antibodies covalently anchored on silicon surfaces via a Si—C bond and cross-linking chemistry.

FIG. 10 shows the chemical structure of the linker.

FIG. 11 illustrates another technique for preparing single pores or periodic pore arrays.

### DETAILED DESCRIPTION OF THE INVENTION

Referring now to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

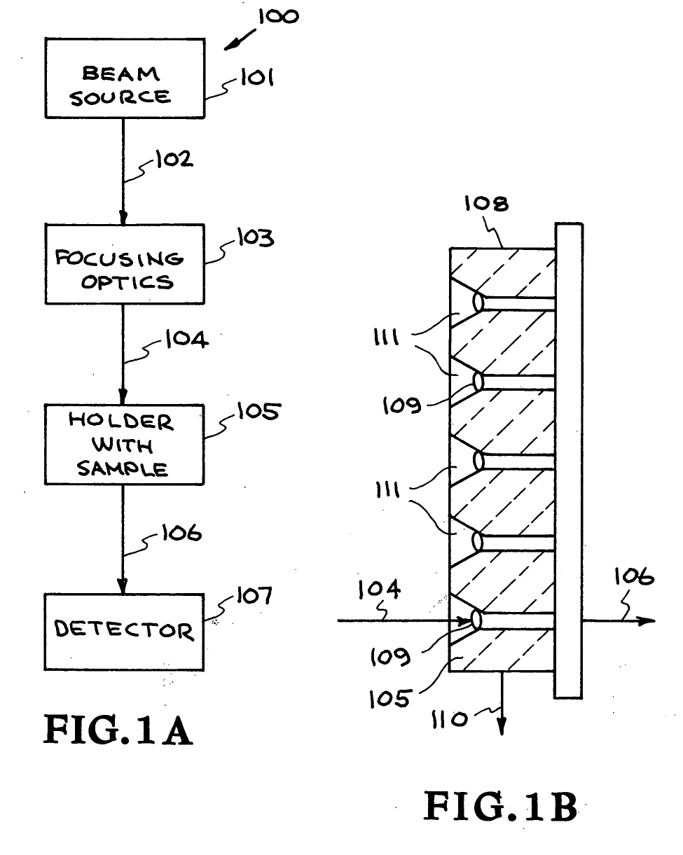

Referring now to the drawings and in particular to FIGS. 1A and 1B, a system for molecule characterization is illustrated. The system is designated generally by the reference numeral 100. As illustrated in FIG. 1A, the system 100 includes: a beam source 101, a system 103 for focusing the beam 102, a focused beam 104, a holder with sample 105, a pattern 105, and a detector 106. The beam source 101 can be a source for producing a beam, a source for producing a pulsed beam, a source for producing a beam of x-rays, a source for producing a beam of x-ray pulses, a source for producing a beam of neutrons, a source for producing a beam of electrons, or a source for producing other beam. For example, the source 101 can be an X-ray tube for diffraction, an x-ray synchrotron, an x-ray free electron laser, a linac, a linac in conjunction with a short pulse laser, or another type of beam source such as a source for producing a beam of photons for optical measurements or a beam of ions for Time of Flight Secondary ion Mass Spectrometry (ToF-SIMS) measurements.

The source 101 produces the beam 102. The beam 102 is focused by the focusing optics 103 which produces a focused beam 104. The focused beam 104 is directed to the holder with sample 105. The holder with sample 105 will be shown in greater detail in FIG. 1B. The focused beam 104 is directed onto the sample producing the pattern 106. The pattern 106 is recorded by the detector 107. The system 100 can be used to determine the structure of individual proteins and viruses to be determined in the dry as well as in the hydrated native state. This constitutes a tremendous improvement compared to the time-consuming protein or virus crystallization technique which only leads to structures in the solid state.

Referring now to FIG. 1B, the functionalized platform for individual molecule characterization portion of the system for molecule characterization in greater detail. FIG. 1B is a side cross section view of the protein holder 108 with a sample 109 in the hydrated phase moved, as illustrated by the arrows 110, under the focused beam 104. The sample 109 can be any sample to be analyzed. For example the sample 109 can be a single molecule, a biological molecule, a non-biological small sample, a small crystal, a virus, a cell, or other sample. In one embodiment, single proteins are collected at the bottom of each functionalized well 111 and excess material is flushed away. A periodic array of single proteins in the hydrated phase remains and can be moved under the beam 104 in synchronization with the beam pulse frequency by using an automated stage.

Referring again to FIG. 1A, a specific embodiment of a system 100 includes: a source 101 of an x-ray beam 102, a system 103 for focusing the x-ray beam 102, a focused x-ray beam 104, a holder with sample 105, an x-ray pattern 105, and a detector 106. The source of x-ray pulses 101 can be any source of x-rays. For example, the 101 can be an X-ray tube for diffraction, an x-ray synchrotron, or another source of x-rays.

The source 101 produces the x-ray beam 102. The x-ray beam 102 is focused by the focusing optics 103 which produces a focused x-ray beam 104. The focused x-ray beam 104 is directed to the holder with sample 105. The holder with sample 105 will be shown in greater detail in FIG. 1B. The focused x-ray beam 104 is directed onto the sample producing the x-ray pattern 106. The x-ray pattern 106 is recorded by the detector 107. The system 100 can be used to determine the structure of individual proteins and viruses to be determined in the dry as well as in the hydrated native state. This constitutes a tremendous improvement compared to the time-consuming protein or virus crystallization technique which only leads to structures in the solid state.

Referring again to FIG. 1B, the functionalized platform for individual bio-molecule characterization portion of the system for bio-molecule characterization in greater detail. FIG. 1B is a side cross section view of the protein holder 108 with a sample 109 in the hydrated phase moved, as illustrated by the arrows 110, under the focused X-ray beam 104. The sample 109 can be any sample to be analyzed. For example the sample 109 can be a single molecule, a biological molecule, a non-biological small sample, a small crystal, or other sample. Single proteins are collected at the bottom of each functionalized well 11 and excess material is flushed away. A periodic array of single proteins in the hydrated phase remains and can be moved under the X-ray beam 104 in synchronization with the beam pulse frequency by using an automated stage.

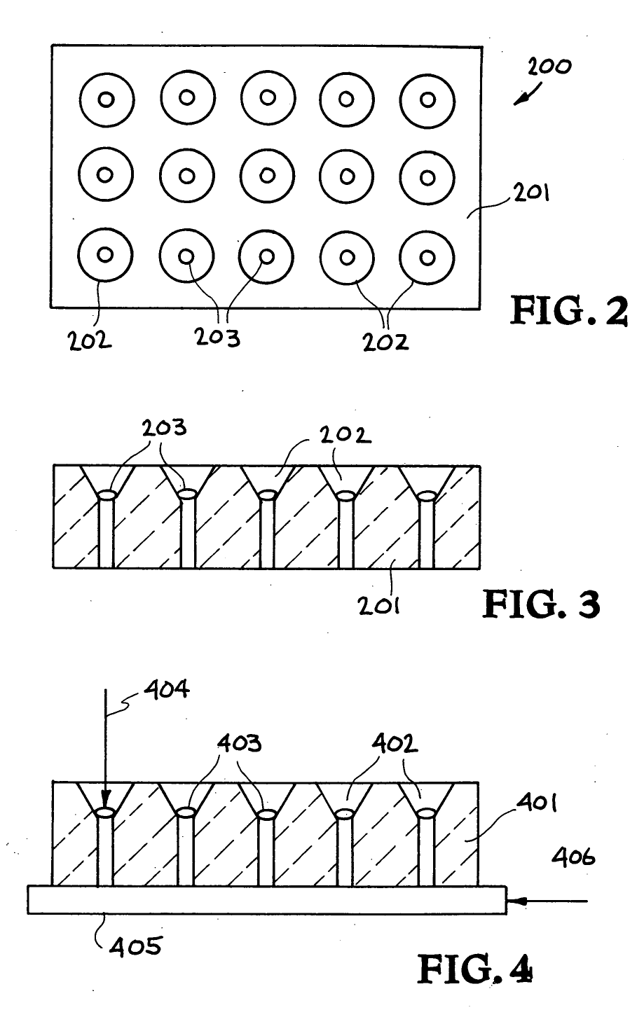

Referring now to FIGS. 2-9, embodiments of the present invention are shown that utilize a periodic array of through holes fabricated on a rigid silicon platform and functionalized in order to accommodate one bio-molecule per pore. The present invention provides a ‘bio-molecule holder’ that will allow the structure of individual proteins and viruses to be determined in dry as well as in the hydrated native state. The present invention constitutes a tremendous improvement compared to the time-consuming protein or virus crystallization technique which only leads to structures in the solid state.

Referring now to FIGS. 2 and 3, a protein holder constructed in accordance with the present invention is illustrated. The protein holder is designated generally by the reference numeral 200. FIG. 2A is a top view of the protein holder 200 and FIG. 3 is a side cross section view of the protein holder 200. The rigid platform 201 for the protein holder 200 is prepared by a combination of micro- and nano-fabrication techniques (including Focused Ion Beam Machining) and/or electrochemistry and its surface is chemically functionalized via cross-linking techniques (a large variety of chemical or physical anchoring techniques are available). The size of the pores 202 is controlled from the nanometer to tens of micrometer regime in order to match the size of the bio-molecule, virus, or cell 203 of interest. Precise chemical functionality in each pore 202 is achieved by a combination of nitride masking, ion-beam-assisted silicon oxide growing, and AFM writing at the pore entrance in order to create an anchor point for a single molecule. Orientation of the molecules 203 on the holder 200 will be controlled by using their polarization properties in an electric field (or in a laser beam) therefore creating a pseudo-crystal.

As illustrated in FIGS. 2 and 3, the present invention provides a periodic array of through holes 202 fabricated on a rigid platform 201 and chemically functionalized in order to accommodate one bio-molecule 203 per pore. This new ‘bio-molecule holder’ 200 will allow the structure of individual proteins and viruses to be determined in a dry as well as in the hydrated native state. The bio-molecule holder 200 provides a tremendous improvement compared to the time-consuming protein or virus crystallization technique which can only be utilized with structures in the solid state.

The silicon platform 201 is prepared by a combination of micro- and nano-fabrication techniques (including Focused ion Beam Machining) and/or electrochemistry and its surface is functionalized via cross-linking techniques. Pore size is controlled from the nanometer to tens of micrometer regime in order to match the size of the bio-molecule of interest. Precise chemical functionality in each pore is achieved by a combination of nitride masking, ion-beam-assisted oxide growing, and AFM writing at the pore entrance in order to create an anchor point for a single molecule. Orientation of the molecules 202 on the holder 201 is controlled by using their polarization properties in an electric field (or in a laser beam) therefore creating a pseudo-crystal.

Referring now to FIG. 4, a side cross section view of an embodiment of a system for bio-molecule characterization constructed in accordance with the present invention is illustrated. The system for bio-molecule characterization is designated generally by the reference numeral 400. The system 400 comprises a protein holder 401 with hydrated phase remains 403 moved under an X-ray beam 404. Single proteins 403 are collected at the bottom of each functionalized well 402 and excess material is flushed away. A periodic array of single proteins in the hydrated phase and can be moved, as illustrated by the arrows 406, under the X-ray beam 404 in synchronization with the beam pulse frequency by using an automated stage 405.

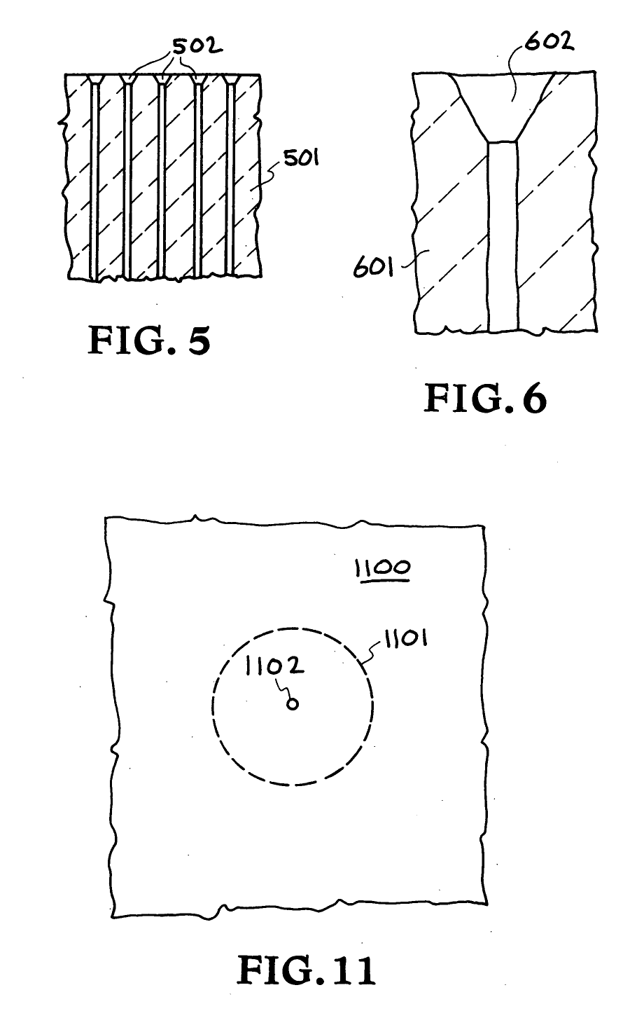

Silicon Platform Preparation: The present invention provides periodic silicon membranes with pore diameters ranging from hundreds of nanometers to tens of microns, suitable for cell, spore, bacteria and large virus capture. The silicon membranes can be prepared by light-assisted electrochemical dissolution of pre-patterned silicon wafers in hydrofluoric acid. This is illustrated in FIGS. 5 and 6. Smaller pore diameters required for protein and small virus capture can also be prepared by changing the electrochemical conditions during the silicon etching process. Data obtained on patterned silicon samples etched by the breakdown technique show that through pores with a top diameter of a few tens of nanometers and aspect ratios up to many hundreds are achievable.

As illustrated in FIGS. 5 and 6, silicon membranes can be prepared by light-assisted electrochemical dissolution of pre-patterned silicon wafers in hydrofluoric acid. FIG. 5 shows a FESEM cross section of a pore 502 having a 500 nm pore diameter in a silicon membrane 501. FIG. 6 shows a close up on one pore 602 in a silicon membrane 601.

These silicon devices are extremely versatile and all their physical parameters can be tuned: the periodicity is given by the pre-patterning top mask, the pore diameter depends on the electrochemical etching conditions and the pore length is controlled by the duration of the KOH etch or of the Deep Reactive Ion Etch during the wafer back patterning.

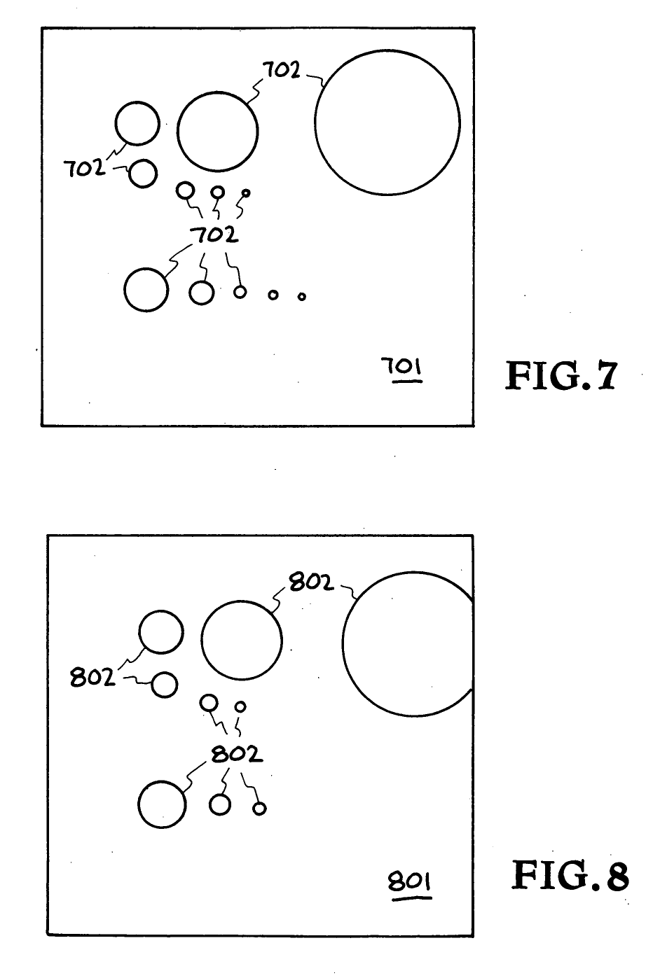

Another technique to prepare periodic pore arrays is Focused Ion Beam (FIB) drilling. Referring now to FIG. 7, a top view of various through apertures 702 prepared by FIB drilling in a silicon membrane 701 is illustrated. Referring to FIG. 8, a top view of various through apertures 802 prepared by FIB drilling in a silicon membrane 801 is shown. Various pore sizes can be achieved from tens of microns down to a few nanometers as shown in FIGS. 7 and 8. The various pore sizes illustrated in FIGS. 7 and 8 can be used for isolating samples in the pore. For example, cells can be isolated in a pore with a pore size of substantially 10 μm. Bacteria can be isolated in a pore with a pore size of substantially 1 μm. Viruses can be isolated in a pore with a pore size of substantially 50 nm. Proteins can be isolated in a pore with a pore size of substantially 5 nm. DNA can be isolated in a pore with a pore size of substantially 2 nm.

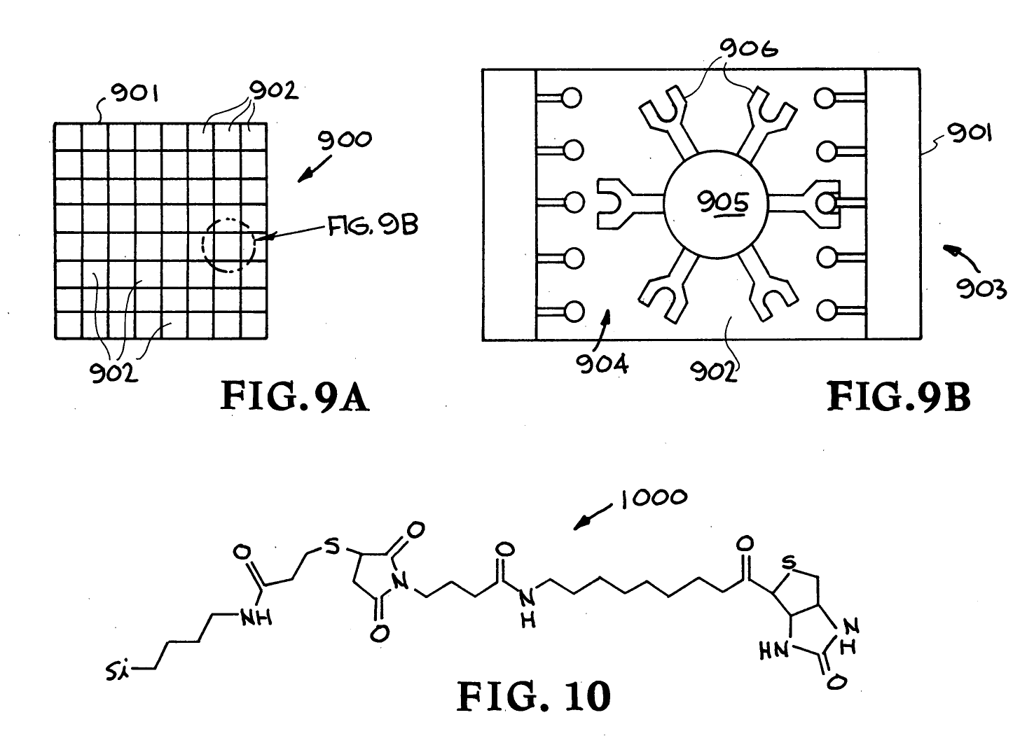

Silicon Membrane Functionalization: Referring now to FIGS. 9A, 9B and 10. FIG. 9A shows pores 902 as a grid structure on substrate 901. FIG. 9B is a greatly enlarged depiction of one of the pores 902 of structure 901. The illustration shows an antibody 905, such as biotin, covalently anchored on a silicon surface 901 in a pore 902. The illustration is designated generally by the reference numeral 900. In the illustration 900, an enlarged section 903 of the silicon surface 901 is shown. Applicants have determined that antibodies such as biotin 905 can be covalently anchored on the silicon surface 901 via a Si—C bond 906 and cross-linking chemistry. Applicants have determined that the same linking system 906 can also be used to anchor larger proteins such as enzymes while preserving enzymatic activity. This attachment technique, as well as the standard cross-linking procedure starting on silanized silicon surfaces, can be used to functionalize the entrance 904 of the silicon holder pore 902. FIG. 9 shows a cross section of a biotin-functionalized silicon membrane 901 used to capture streptavidin-coated 200 nm diameter micro-beads 905. FIG. 10 shows the chemical structure of the linker 1000 used. This linker structure is an example, shorter or longer linkers can be used at will.

Bio-molecule Immobilization: In order to only functionalize the entrance of the pore to immobilize a single protein, a combination of nitride masking, ion-beam-assisted oxide growth, and AFM writing will be used. Using the fact that proteins can be polarized in an electric field or in a laser beam, the proteins will be deposited from solution onto the silicon holder in a unique orientation and attached to the surface of the functionalized silicon surface. A two dimensional array of ordered proteins created in this manner will allow one to determine structure without need for crystallization. This pseudo-protein crystal can then be used to determine the structure of the many types of protein that cannot be crystallized in the conventional manner.

The flow-through configuration of the device will enhance the probability of capturing proteins and will allow a capture control by current-blockade measurements. The ionic current through the device will be measured and will diagnose the presence of open pores.

Device Utilization and Structure Determination: As best illustrated in FIG. 4, the protein holder 401 is mounted on an automated XY stage 405 moving at the frequency of the X-ray, electron or neutron pulses and each pulse will diffract on a single or a small cluster of proteins 403. This configuration enables new experiments to be performed.

Examples of applications include, but are not limited to:
Crystallographic structure of proteins and viruses in either the dry or hydrated state.
Investigation of the effect of a single or of multiple linkers on protein conformation.
Investigation of the effect of solution parameters such as pH and salt concentration on protein conformation.
In situ binding experiments on systems such as ssb (single strand DNA binding) protein—DNA
Investigation of protein complex formation

Single bio-molecule assay: The silicon holder 401 will also insure that a single protein, virus, bacterium, spore or cell 403 is immobilized in one pore 402. It therefore constitutes a platform to study the properties of these biological objects one at a time. It also allows the experimenter to come back to a specified pore (i.e., object of interest) if desired, which is not trivial while working in the liquid phase with free floating organisms.

Examples of characterization techniques that can be coupled to the silicon holder 401 include:
Optical and electronic microscopy
Luminescence
Electrochemistry
Current blockade measurements and Coulter-counting
Secondary Ion Mass Spectrometry (SIMS, ToF SIMS, Nano-SIMS)
Energy Dispersive X-ray Spectroscopy (EDS)

Referring now to FIG. 11, another technique for preparing periodic pore arrays is illustrated. A pore is produced in a sample holder 1100 for holding the sample by forming an aperture and sizing the aperture with localized oxide. Initially a starting through-pore aperture 1101 is produce that extends through the silicon body of the sample holder 1100. The through-pore aperture 1101 has a starting aperture with a diameter of 250 nm. The starting through-pore aperture 1101 is sized by reducing the diameter with localized TEOS oxide to produce a final through-pore aperture 1102. The final through-pore aperture 1102 has a diameter of 38 nm. The final through-pore aperture 1102 is produced by oxide grown by ion-beam assisted technique.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. An apparatus for characterization of a sample, comprising:
a source for directing a beam onto the sample to produce energy emanating from the sample,
a sample holder,
at least one pore in said sample holder for holding the sample, and
a detector for detecting said energy emanating from the sample.

2. The apparatus for characterization of a sample of claim 1 wherein said at least one pore in said sample holder is a nano-size pore.

3. The apparatus for characterization of a sample of claim 1 wherein said at least one pore in said sample holder is a micro-size pore.

4. The apparatus for characterization of a sample of claim 1 wherein said at least one pore in said sample holder is a pore that holds a single molecule.

5. The apparatus for characterization of a sample of claim 1 wherein said at least one pore in said sample holder is a pore that holds a biological molecule.

6. The apparatus for characterization of a sample of claim 1 wherein said at least one pore in said sample holder is a pore that holds a non-biological sample.

7. The apparatus for characterization of a sample of claim 1 wherein said at least one pore in said sample holder is a pore that holds a small crystal.

8. The apparatus for characterization of a sample of claim 1 wherein said at least one pore in said sample holder is a pore that holds a protein.

9. The apparatus for characterization of a sample of claim 1 wherein said at least one pore in said sample holder is a pore that holds a virus.

10. The apparatus for characterization of a sample of claim 1 wherein said at least one pore in said sample holder is a pore that holds a pseudo-protein crystal.

11. The apparatus for characterization of a sample of claim 1 wherein said at least one pore in said sample holder holds the sample in a hydrated state.

12. The apparatus for characterization of a sample of claim 1 wherein said at least one pore in said sample holder is a pore that holds a cell.

13. The apparatus for characterization of a sample of claim 1 wherein said at least one pore in said sample holder is a pore that holds a spore.

14. The apparatus for characterization of a sample of claim 1 wherein said at least one pore in said sample holder for holding the sample is a through-pore that extends through said sample holder.

15. The apparatus for characterization of a sample of claim 1 wherein said at least one pore in said sample holder for holding the sample is a through-pore that extends through said sample holder and said through-pore is an aperture with localized oxide.

16. The apparatus for characterization of a sample of claim 1 wherein said at least one pore in said sample holder for holding the sample is a through-pore that extends through said sample holder and said through-pore is an aperture with localized TEOS oxide.

17. The apparatus for characterization of a sample of claim 1 wherein said beam is an x-ray beam.

18. The apparatus for characterization of a sample of claim 1 wherein said diffractive beam is a pulsed x-ray beam.

19. The apparatus for characterization of a sample of claim 1 wherein said diffractive beam is a neutron beam.

20. The apparatus for characterization of a sample of claim 1 wherein said diffractive beam is an electron beam.

21. The apparatus for characterization of a sample of claim 1 wherein said diffractive beam is a photon beam.

22. The apparatus for characterization of a sample of claim 1 wherein said diffractive beam is a photon beam for optical experiments.

23. The apparatus for characterization of a sample of claim 1 wherein said diffractive beam is an ion beam.

24. The apparatus for characterization of a sample of claim 1 wherein said diffractive beam is an ion beam for SIMS measurements.

25. The apparatus for characterization of a sample of claim 1 wherein said sample holder comprises a silicon platform.

26. The apparatus for characterization of a sample of claim 1 wherein said sample holder comprises a rigid silicon platform.

27. The apparatus for characterization of a sample of claim 1 including means for moving said sample holder.

28. The apparatus for characterization of a sample of claim 1 including a linking system connected to said at least one pore in said sample holder for anchoring the sample in said at least one pore.

29. The apparatus for characterization of a sample of claim 1 including a cross-linking system connected to said at least one pore in said sample holder for anchoring the sample in said at least one pore.

30. The apparatus for characterization of a sample of claim 1 including a cross-linking system connected to said at least one pore in said sample holder for anchoring larger proteins in said at least one pore.

31. The apparatus for characterization of a sample of claim 1 including a cross-linking system connected to said at least one pore in said sample holder for anchoring enzymes in said at least one pore.

32. The apparatus for characterization of a sample of claim 1 including a covalent anchor connected to said at least one pore in said sample holder.

33. An apparatus for characterization of a sample, comprising:
source means for directing a beam onto the sample to produce energy emanating from the sample,
sample holder means,
pore means in said sample holder means for holding the sample, and
detector means for detecting said energy emanating from the sample.

34. The apparatus for characterization of a sample of claim 33 wherein said source means is a source for producing an x-ray beam and said energy emanating from the sample is a diffraction pattern.

35. The apparatus for characterization of a sample of claim 33 wherein said source means is a source for producing a pulsed x-ray beam and said energy emanating from the sample is a diffraction pattern.

36. The apparatus for characterization of a sample of claim 33 wherein said source means is a source for producing a neutron beam.

37. The apparatus for characterization of a sample of claim 33 wherein said source means is a source for producing an electron beam.

38. The apparatus for characterization of a sample of claim 33 wherein said source means is a source for producing a photon beam.

39. The apparatus for characterization of a sample of claim 33 wherein said source means is a source for producing an ion beam.

40. The apparatus for characterization of a sample of claim 33 including means for moving said sample holder means.

41. The apparatus for characterization of a sample of claim 1 wherein said pore means for holding the sample is a through-pore that extends through said sample holder means.

42. The apparatus for characterization of a sample of claim 1 wherein said pore means for holding the sample is a through-pore that extends through said sample holder means and said through-pore is an aperture with localized oxide.

43. The apparatus for characterization of a sample of claim 1 wherein said pore means for holding the sample is a through-pore that extends through said sample holder means and said through-pore is an aperture with localized TEOS oxide.

44. The apparatus for characterization of a sample of claim 33 including a linking system connected to said pore means in said sample holder means for anchoring the sample in said pore means.

45. The apparatus for characterization of a sample of claim 33 including a cross-linking system connected to said pore means in said sample holder means for anchoring the sample in said pore means.

46. The apparatus for characterization of a sample of claim 33 including a cross-linking system connected to said pore means in said sample holder means for anchoring larger proteins in said pore means.

47. The apparatus for characterization of a sample of claim 33 including a cross-linking system connected to said pore means in said sample holder means for anchoring enzymes in said pore means.

48. The apparatus for characterization of a sample of claim 33 including a covalent anchor connected to said pore means in said sample holder.

49. A method of characterization of a sample, comprising the steps of:
producing a beam,
providing a sample holder,
providing at least one through-pore in said sample holder that extends through said sample holder,
positioning the sample in said sample holder,
positioning said beam and said sample holder so that the sample receives said beam, and
detecting energy released from the sample.

50. The method for characterization of a sample of claim 49 wherein said step of providing at least one through-pore in said sample holder comprises providing a nano-size through-pore in said sample holder.

51. The method for characterization of a sample of claim 49 wherein said step of providing at least one through-pore in said sample holder comprises providing a micro-size through-pore in said sample holder.

52. The method for characterization of a sample of claim 49 wherein said step of positioning the sample in said sample holder comprises positioning a single molecule in said through-pore in said sample holder.

53. The method for characterization of a sample of claim 49 wherein said step of positioning the sample in said sample holder comprises positioning a biological molecule in said through-pore in said sample holder.

54. The method for characterization of a sample of claim 49 wherein said step of positioning the sample in said sample holder comprises positioning a non-biological molecule in said through-pore in said sample holder.

55. The method for characterization of a sample of claim 49 wherein said step of positioning the sample in said sample holder comprises positioning a small crystal in said through-pore in said sample holder.

56. The method for characterization of a sample of claim 49 wherein said step of positioning the sample in said sample holder comprises positioning a protein in said through-pore in said sample holder.

57. The method for characterization of a sample of claim 49 wherein said step of positioning the sample in said sample holder comprises positioning a virus in said through-pore in said sample holder.

58. The method for characterization of a sample of claim 49 wherein said step of positioning the sample in said sample holder comprises positioning a pseudo-protein crystal in said through-pore in said sample holder.

59. The method for characterization of a sample of claim 49 wherein said step of positioning the sample in said sample holder comprises positioning a cell in said through-pore in said sample holder.

60. The method for characterization of a sample of claim 49 wherein said step of producing a beam comprises producing an x-ray beam.

61. The method for characterization of a sample of claim 49 wherein said step of producing a beam comprises producing a pulsed x-ray beam.

62. The method for characterization of a sample of claim 49 wherein said step of producing a beam comprises producing a neutron beam.

63. The method for characterization of a sample of claim 49 wherein said step of directing a beam onto the sample comprises directing an electron beam onto the sample.

64. The method for characterization of a sample of claim 49 wherein said step of directing a beam onto the sample comprises directing a proton beam onto the sample.

65. The method for characterization of a sample of claim 49 wherein said step of directing a beam onto the sample comprises directing an ion beam onto the sample.

66. The method for characterization of a sample of claim 49 wherein said step of providing at least one pore in said sample holder comprises providing at least one pore in a silicon platform.

67. The method for characterization of a sample of claim 49 wherein said step of providing at least one pore in said sample holder comprises providing at least one pore in a rigid silicon platform.

68. The method for characterization of a sample of claim 49 wherein said step of providing at least one pore in said sample holder includes providing a linking system connected to said at least one pore in said sample holder for anchoring the sample in said at least one pore.

69. The method for characterization of a sample of claim 49 wherein said step of providing at least one pore in said sample holder includes providing a cross-linking system connected to said at least one pore in said sample holder for anchoring the sample in said at least one pore.

70. The method for characterization of a sample of claim 49 wherein said step of providing at least one pore in said sample holder includes providing a cross-linking system connected to said at least one pore in said sample holder for anchoring larger proteins in said at least one pore.

71. The method for characterization of a sample of claim 49 wherein said step of providing at least one pore in said sample holder includes providing a cross-linking system connected to said at least one pore in said sample holder for anchoring enzymes in said at least one pore.

72. The method for characterization of a sample of claim 49 wherein said step of providing at least one pore in said sample holder includes providing a covalent anchor connected to said at least one pore in said sample holder.

73. The method for characterization of a sample of claim 49 wherein said step of providing at least one pore in said sample holder includes providing at least one pore in said sample holder that holds the sample in a hydrated state.

74. A sample holder apparatus adapted for characterization of a sample using a beam, comprising:
a rigid platform having a first side and a second side, and
a through-pore in said rigid platform that extends through said rigid platform from first side to said second side, wherein said through-pore being is of size that holds a single molecule sample or a single cell sample, and wherein said through-pore has an entrance on said first side for receiving said single molecule sample or a single cell sample and the beam and an exit on said second side.

75. The sample holder apparatus of claim 74 wherein said through-pore is a nano-size through-pore.

76. The sample holder apparatus of claim 74 wherein said through-pore is a micro-size through-pore.

77. The sample holder apparatus of claim 74 wherein said through-pore is an aperture with localized oxide.

78. The sample holder apparatus of claim 74 wherein said through-pore is an aperture with localized TEOS oxide.

79. The sample holder apparatus of claim 74 including a linking system connected to said through-pore for anchoring the sample in said through-pore.

80. The sample holder apparatus of claim 74 including means for moving said rigid platform.

81. The sample holder apparatus of claim 74 wherein said rigid platform is a rigid silicon platform.

82. The sample holder apparatus of claim 74 wherein said rigid platform is a rigid glass platform.
